(19) United States
(12) Patent Application Publication
(10) Pub. No.: US 2004/0059386 A1
(43) Pub. Date: Mar. 25, 2004

(54) Low frequency foot-electric stimulating assistor

(76) Inventor: Tsung-l Yu, Taipei (TW)

Correspondence Address: BACON & THOMAS, PLLC, 625 SLATERS LANE, FOURTH FLOOR, ALEXANDRIA, VA, 22314

(21) Appl. No.: 10/352,144

(22) Filed: Jan. 28, 2003

Related U.S. Application Data
(63) Continuation-in-part of application No. 10/253,817, filed on Sep. 25, 2002.

(51) Int. Cl. A61N 1/00

(52) U.S. Cl. 607/2

(57) ABSTRACT

A low frequency foot-electric stimulating assistor which is an electric stimulating piece comprising at least one pair of positive and negative electrodes connected in series or in parallel and installed within a conductive film made of copper foils or conductive rubber materials. The electric stimulating piece takes the shape of a shoe pad, and the positive and negative electrodes therein are arranged in accordance with the distribution of the acupuncture points on the bottom of a human foot. The electric stimulating piece may embody itself in a shoe pad, which then integrated in a shoe. A controller controls the functioning of the electric stimulating piece. By controlling the emission of low-frequency electromagnetic waves at chosen positive and negative electrodes, an effect of electrically stimulated massage that can promote health is achieved.

The present invention is a continuation in part (CIP) of U.S. patent series No. 10/253,817, filed at Sep. 25, 2002, which is incorporated into the present invention as a part of the present invention.

### BACKGROUND OF THE INVENTION

(a) Field of the Invention

The present invention relates to a low frequency foot-electric stimulating assistor, more particular to an electric stimulating piece comprising a conductive film made of copper foils or conductive rubber materials and at least a pair of positive and negative electrodes embedded therein; the electrodes are connected either in series or in parallel. Activated by a controller, a number of electrode pairs can be chosen to emit low-frequency electromagnetic waves, and therefore the electric stimulating piece, taking the shape of a shoe pad, can be applied to provide an electrically stimulated massage via low-frequency waves to the acupuncture points on the bottom of a human foot.

(b) Description of the Prior Art

The foot massaging devices of the prior art usually adopt a mechanism of an eccentric wheel or a foot stepping pad having granules formed thereon to produce a massaging effect. In close contact with a foot as the foot is pressed on, those devices stimulate the acupuncture points on the bottom of a human foot. In spite of structural simplicity, the massaging effect is less prominent.

### SUMMARY OF THE INVENTION

The purpose of the present invention is for providing a low frequency foot-electric stimulating assistor, which is an electric stimulating piece comprising a conductive film made of copper foils or conductive rubber materials and at least a pair of positive and negative electrodes embedded therein; the electrodes are connected either in series or in parallel. Activated by a wired or wireless controller, a number of electrode pairs can be chosen to emit low-frequency electromagnetic waves. When a foot rest passively on the electric stimulating piece, those activated electrode pairs can electrically stimulate the foot acupuncture points they correspond to, achieving a massaging effect that could promote the blood circulation therein.

More particularly, the present invention of a low frequency foot-electric stimulating assistor can embody itself in a shoe pad of a shoe, on which the positive and negative electrodes are distributed according to the acupuncture points in the reflex areas on the bottom of a human foot. Activated by a controller, a number of electrode pairs can be chosen to provide low frequency electrically stimulated massage to the corresponding acupuncture points, which may promote health.

### BRIEF DESCRIPTION OF THE DRAWINGS

FIG. 1 illustrates a partial section of an electric stimulating piece composed of at least one pair of positive and negative electrodes, connected in series or in parallel, embedded in a conductive film.

FIG. 2 is a cross-sectional view of the electric stimulating piece illustrated in FIG. 1.

FIG. 3 is a perspective view of the controller used with the electric stimulating piece.

FIG. 4 illustrates the acupuncture points on the bottom of a human foot.

FIG. 5 is a top view of an embodiment of the present invention as a shoe-pad electric stimulating piece.

FIG. 6 is a perspective view of an embodiment of the present invention as a shoe-pad electric stimulating piece integrated in a shoe.

FIG. 7 is a perspective view of an,embodiment of the present invention in which two electric stimulating pieces integrated in a massage treading pad.

FIG. 8 shows an embodiment of the present invention.

FIG. 9 shows another embodiment of the present invention.

FIG. 10 shows a yet embodiment of the present invention.

FIG. 11 shows a further embodiment of the present invention.

FIG. 12 is a perspective view of another embodiment of the present invention applied to a shoe.

FIG. 13 is a perspective view of another embodiment of the present invention applied to a shoe.

FIG. 14 is a perspective view of another embodiment of the present invention applied to a shoe.

FIG. 15 is a perspective view of another embodiment of the present invention in which two electric stimulating pieces integrated in a massage treading pad

### DETAILED DESCRIPTION OF THE PREFERRED EMBODIMENTS

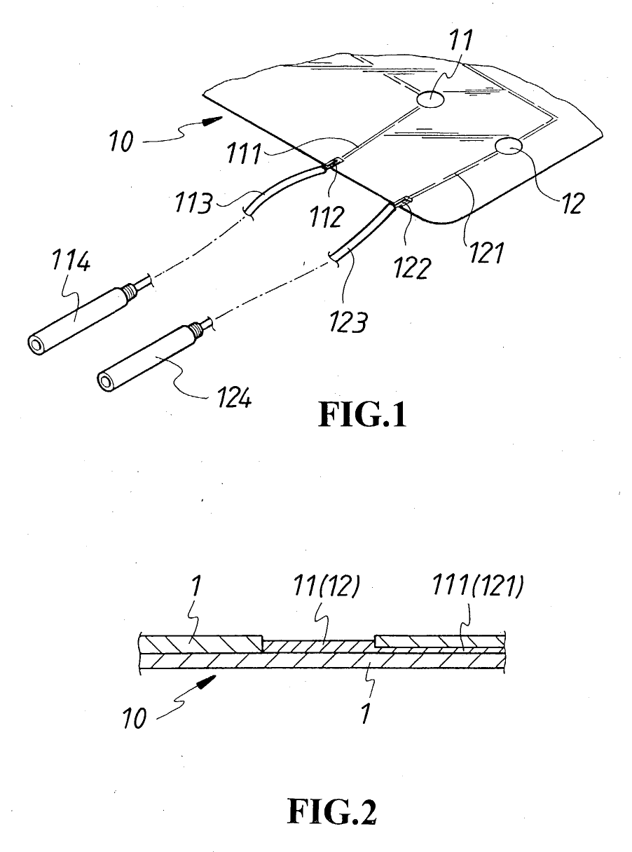

As shown in FIGS. 1 and 2, this present invention as a low frequency foot-electric stimulating assistor in which at least one pair of positive and negative electrodes 11 and 12 is built into a conductive film 1, made of copper foils or conductive rubber materials, and connected in series or in parallel to form an electric stimulating piece 10. In more detail, the positive and negative electrodes 11 and 12 are respectively connected to electrode joints 112 and 122 through conductive paths 111 and 121. The electrode joints 112 and 122 are further respectively connected to conductive inserting pins 114 and 124 through wires 113 and 123. With a distance apart, the conductive paths 111 and 121 can be fixed within the conductive film 1 between two thin foils so that the positive and negative electrodes 11 and 12 are in a state of insulation.

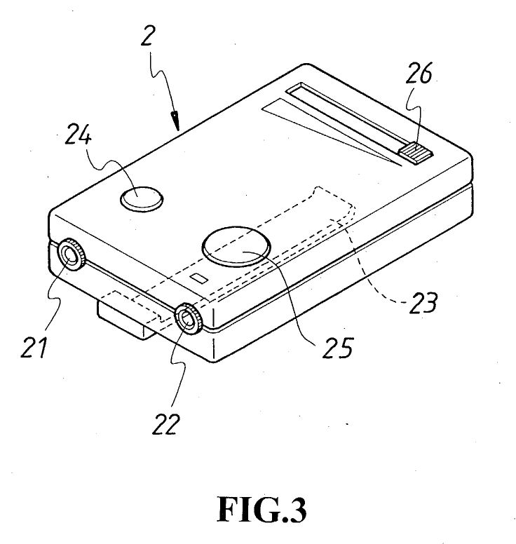

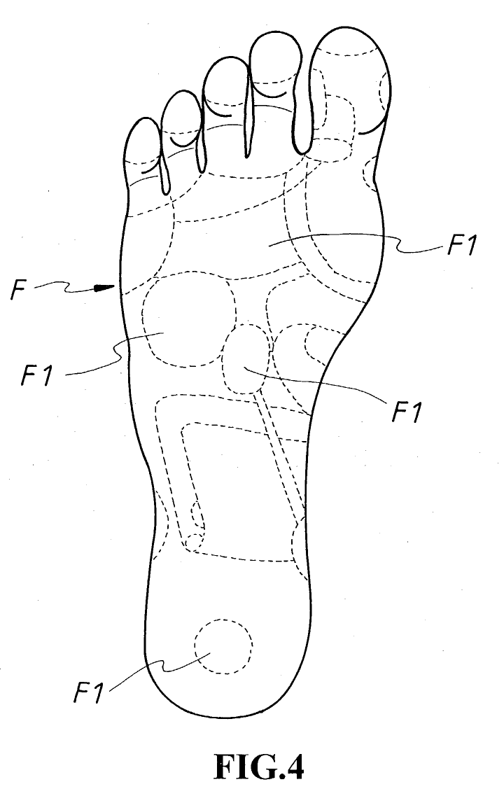

The above-mentioned electric stimulating piece 10, composed of at least one pair of the positive and negative electrodes 11 and 12 embedded in a conductive film 1 (made of copper foils or conductive rubber materials), is for a foot to rest passively on in a position that the foot acupuncture points F1 align with the positive and negative electrodes 11 and 12. Meanwhile, low frequency electromagnetic waves are activated, at the pairs of positive and negative electrodes 11 and 12 by a wired or wireless controller 2 as shown in FIG. 3, to stimulate electrically the area about an acupuncture point F1 as shown in FIG. 4. The electric stimulation provides an effect of massage that improves the blood circulation in the body portion the electric stimulating piece 10 is pressed on, eventually leading an effect of promoting one's health. The functions of the above-mentioned controller 2 are at least switching on/off an output current and regulating the magnitude of the current. Control switches 24 and 25 are for starting and stopping the control and an adjusting switch 26 is for regulating the output current; the adjusting switch 26 controls a stepless adjustment by a knob or a push-pull piece. The controller 2 contains batteries and the conductive inserting pins 21 and 22 thereof are connected to the conductive inserting pins 114 and 124 of the electric stimulating piece 10. Further, a clip 23 can be installed at a chosen area on the controller 2 for wearing on clothes, if desired.

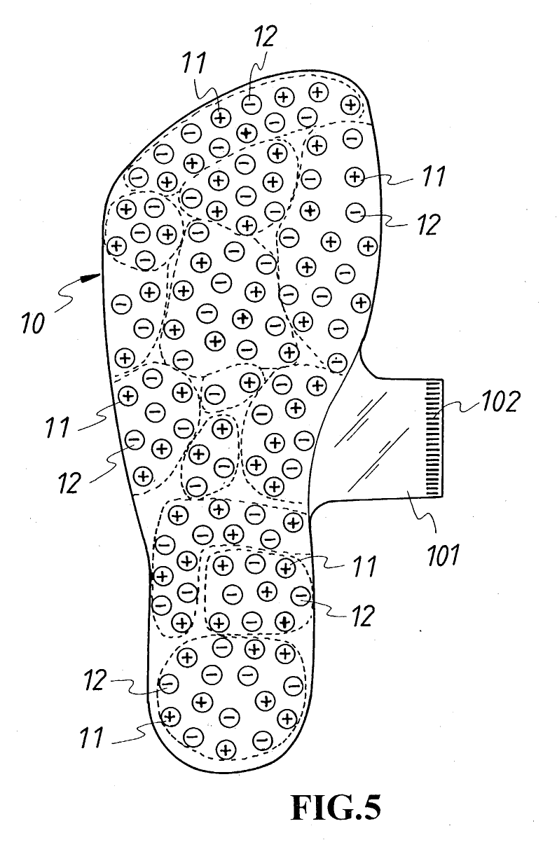

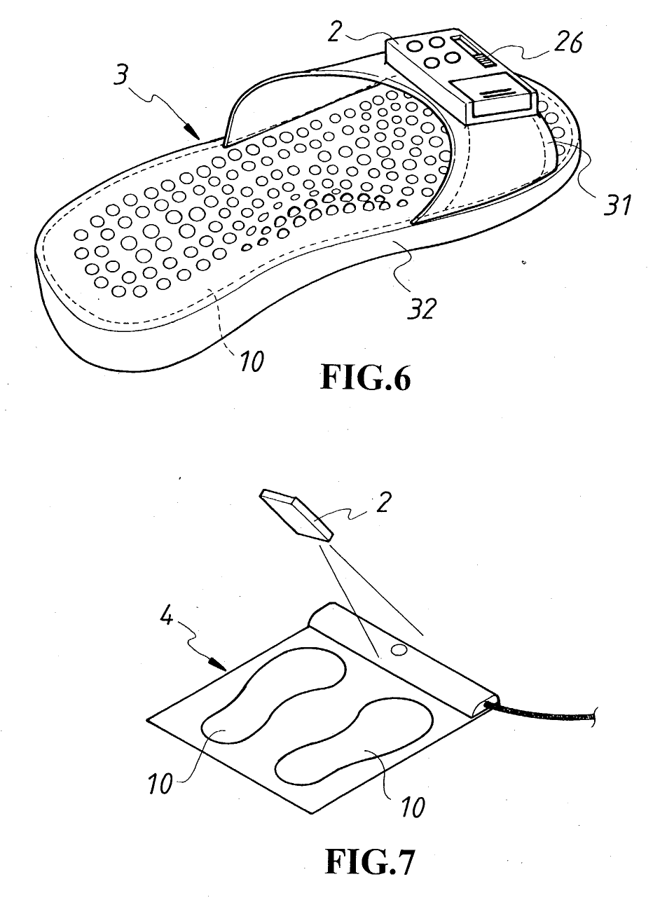

As shown in FIG. 5, an embodiment of the present invention is an electric stimulating piece 10 in the shape of a shoe pad. One or more pairs of positive and negative electrodes, corresponding to the acupuncture points in various reflex areas on the bottom of a foot, are embedded within the electric stimulating piece 10, connected either in parallel or in series. The wires connected to those electrodes are converged to a lateral piece 101 to form a BUS wire portion 102, which portion is for connecting to a wired controller 2.

The shoe-pad shaped electric stimulating piece 10 described above can be attached to a shoe to form a combined configuration. Further, a wired or remote controller 2 can be attached to the top wearing piece 31 of the shoe for activating the emission of low frequency electromagnetic waves at electrodes embedded within the electric stimulating piece to achieve an effect of massaging acupuncture points in the reflex areas on the bottom of a human foot.

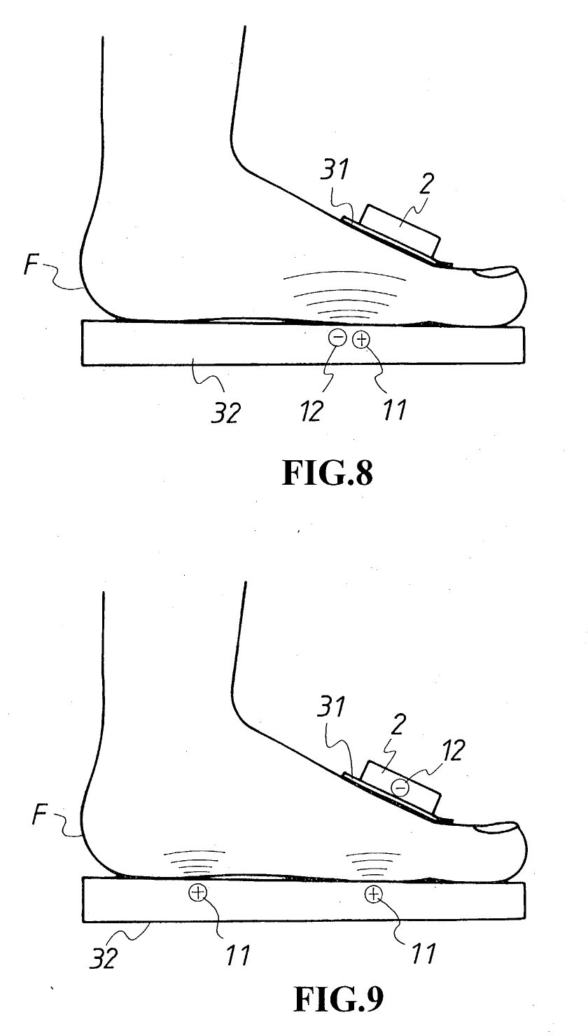

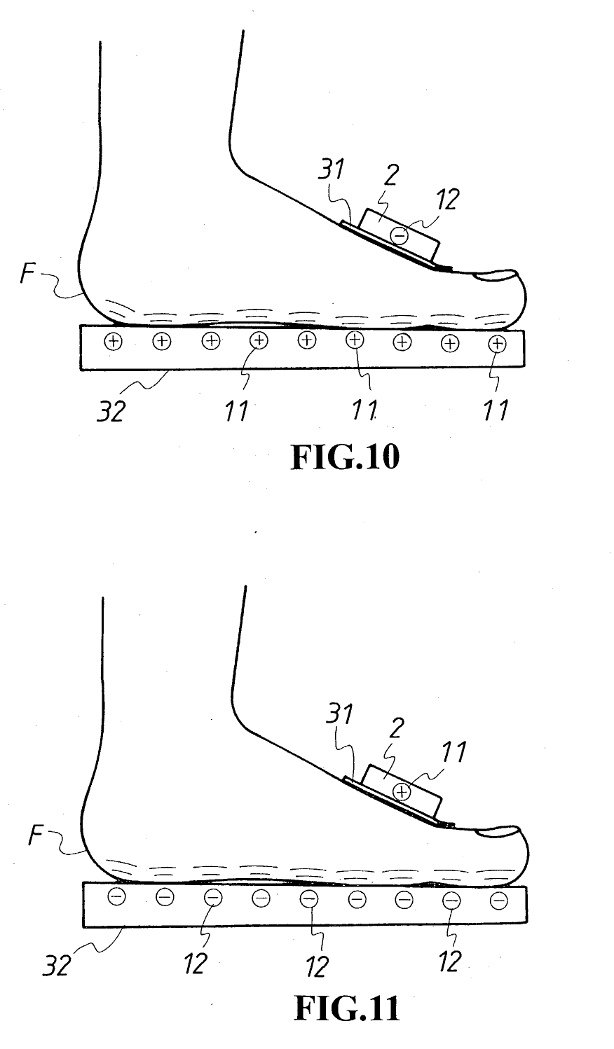

Further, as the present invention embodies itself in a shoe 3, electrodes of one polarity can be installed within the top wearing piece 3 1 while those of the other polarity are installed within the bottom wearing piece 32; the electrodes can be arranged in a concentrated way or a distributed way, depending on actual requirements. The various forms of application are illustrated by the embodiments shown in FIGS. 8 to 11.

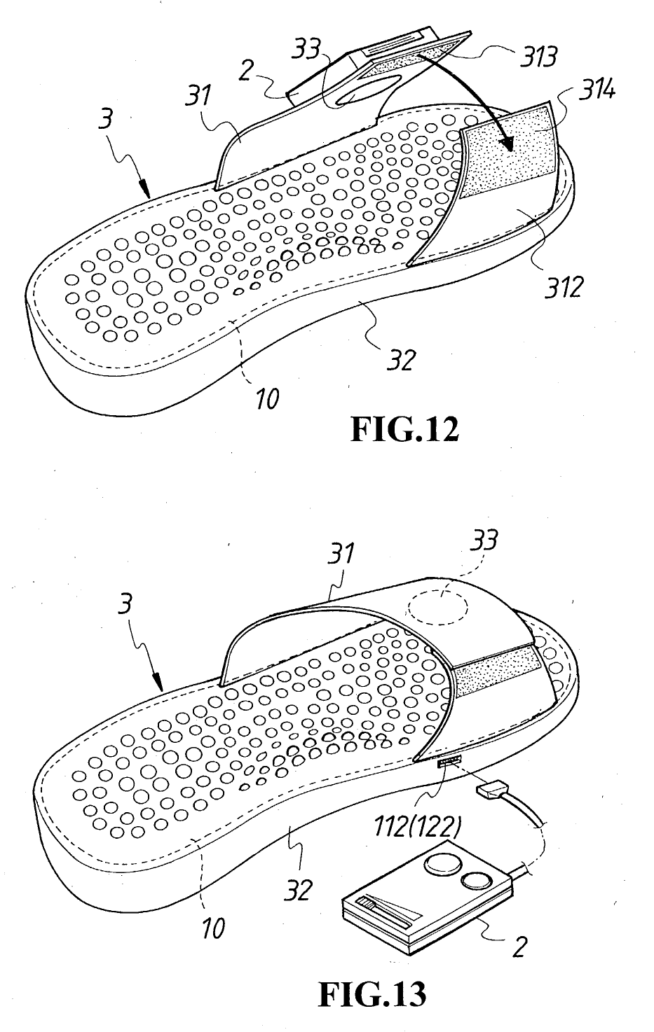

As shown in FIG. 12, as the present invention embodies itself in a shoe 3, an electrode plate of positive or negative polarity 33 can be installed on the inner wall of a top wearing piece 31, the electrode plate 33 being coupled with those electrodes in the shoe-pad shaped electric stimulating piece 10 to create an effect of electrically stimulated massage. Instead of being an integral part, the top wearing piece 31 of the shoe 3 may comprises two separated pieces 311 and 312, which can be connected by the adhesive or buckling-up components 313 and 314 form in the overlapping sections of the separated pieces 311 and 312; in this way the top wearing piece 31 fits foots of various sizes and therefore the electrode plate 33 is in close contact with a foot.

By the same token, as shown in FIG. 13, as the present invention embodies itself in a shoe 3, an electrode joint 112 or 122 of the controller 2 can be installed with in the bottom wearing piece 32 for externally activating low-frequency electrically stimulated massage.

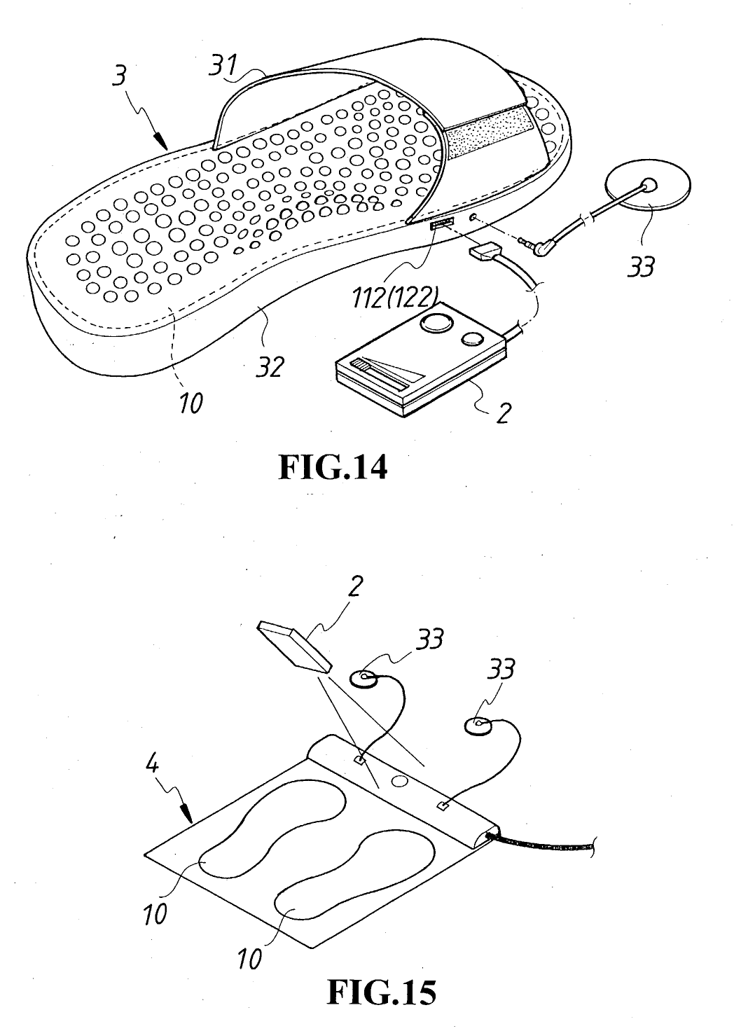

As shown in FIG. 14, the present invention can embody itself in an electrode plate of positive or negative polarity being connected externally by a wire to a shoe 3 at a chosen location so that the electrode plate 33 may stick to the top part of a foot. The configuration works with an external controller 2, wired to the bottom wearing piece 32 of the shoe 3,to achieve low-frequency electrically stimulated massage.

As shown in FIG. 15, the present invention embodies itself in a massage treading pad 4, in which two extended adhesive electrodes are installed on the massage treading pad 4 for sticking to foots for low-frequency electrically stimulated massage, in the same spirit of the above-mentioned embodiments in a shoe 3.

The various application configurations of a low frequency foot-electric stimulating assistor illustrated above are only embodiments of the present invention, which should not restrict the technical field of the present invention. Other equivalent applications made by obvious changes based on the same techniques should be included in the technical field of the present invention.

What is claimed is:

1. A low frequency foot-electric stimulating assistor comprising:
at least one pair of positive and negative electrodes, connected in series or in parallel, and
a conductive film made of copper foils or conductive rubber materials;
said positive and negative electrodes being embedded in said conductive film to define an electric stimulating piece; said electric stimulating piece having the shape of a shoe pad, wherein said positive and negative electrodes are distributed according to the acupuncture points in reflex areas on a bottom of a human foot.

2. The low frequency foot-electric stimulating assistor in claim 1 wherein said positive and negative electrodes are respectively connected to positive and negative electrode joints through conductive paths.

3. The low frequency foot-electric stimulating assistor of claim 2 wherein said positive and negative electrode joints are respectively connected to positive and negative conductive inserting pins by wires.

4. The low frequency foot-electric stimulating assistor of claim 2 wherein said conductive film is composed of two thin foils, and said conductive paths are fixed within said conductive film between said two thin foils to form a state of insulation.

5. The low frequency foot-electric stimulating assistor of claim 1, wherein said assistor is controlled by a wired or wireless controller; said controller loading electric current to a number of pairs of said positive and negative electrodes within said electric stimulating piece.

6. The low frequency foot-electric stimulating assistor of claim 5 wherein said controller switches on/off an output electric current to said positive and negative electrodes within said electric stimulating piece and regulates the magnitude of said electric current; further, said controller having a function of choosing designated pairs of said positive and negative electrodes to load electric current.

7. The low frequency foot-electric stimulating assistor of claim 1, wherein said electric stimulating piece has a shape of a shoe pad, wherein the conductive paths that connect said pairs of positive and negative electrodes converging to a lateral piece to form a BUS wire portion.

8. The low frequency foot-electric stimulating assistor of claim 1, wherein said electric stimulating piece has a shape of a shoe pad, being attached to a shoe to become a shoe pad; the base of said shoe and said attached shoe pad defining a bottom wearing piece and the upper cover of said shoe defining a top wearing piece.

9. The low frequency foot-electric stimulating assistor of claim 1, wherein said electric stimulating piece having a shape of a shoe pad, being integrated into a massage treading pad.

10. The low frequency foot-electric stimulating assistor of claim 8 wherein at least one pair of positive and negative electrodes is installed within each of said top wearing piece and said bottom wearing piece of said shoe; the arrangement of said pairs of positive and negative electrodes are either concentrated or distributed.

11. The low frequency foot-electric stimulating assistor of claim 8 wherein an electrode plate is installed on an inner wall of said top wearing piece.

12. The low frequency foot-electric stimulating assistor of claim 8 wherein said top wearing piece is composed of two separated pieces, said separated pieces having adhesive or buckling-up components in the overlapping sections thereof.

13. The low frequency foot-electric stimulating assistor of claim 8 wherein a controller is attached to said top wearing piece.

14. The low frequency foot-electric stimulating assistor of claim 8 wherein conductive inserting pins of two polarities are installed respectively on said top wearing piece and said bottom wearing piece for connecting a controller and a sticky electrode plate.

15. The low frequency foot-electric stimulating assistor of claim 8 wherein a controller is connected to said bottom wearing piece and a sticky electrode plate is installed on said top wearing piece.

16. The low frequency foot-electric stimulating assistor of claim 9 wherein two sticky electrode plates, extendable by wires, are installed on said massage treading pad.
